(19) United States
(12) Patent Application Publication
(10) Pub. No.: US 2016/0018394 A1
(43) Pub. Date: Jan. 21, 2016

(54) Assay Device Having Multiplexing

(71) Applicant: Ortho-Clinical Diagnostics, Inc., Raritan, NJ (US)

(72) Inventors: Susan Danielson, Honeoye Falls, NY (US); James D. Kanaley, Honeoye Falls, NY (US); Zhong Ding, Pittsford, NY (US); Philip C. Hosimer, Rochester, NY (US); James W. Hassett, Ontario, NY (US); Timothy C. Warren, Rochester, NY (US); David A. Heavner, Fairport, NY (US)

(21) Appl. No.: 14/333,571

(22) Filed: Jul. 17, 2014

(51) Int. Cl. G01N 33/543

(52) U.S. Cl. CPC G01N 33/54386; G01N 33/54306

(57) ABSTRACT

An assay device for determining the concentration of multiple analytes or controls, where the device is capable of determining the presence or concentration of at least six analytes or controls includes: a fluid flow path; a liquid sample addition zone; a reagent zone downstream and in fluid communication with the sample addition zone containing one or more reagents; multiple detection zones in fluid communication with the reagent zone. The fluid flow path, which extends through the detection zones, has a length capable of having at least six detection zones linearly spaced at least a sufficient distance apart in order to discriminate each signal peak from its adjacent signal peak. The device further includes a wicking zone in fluid communication with the detection zones having a capacity to receive liquid sample flowing from the detection zone. The fluid flow path extends from the sample zone to the wicking zone, and at least a part of the fluid flow path has a substrate and projections which extend substantially vertically from the substrate. The projections have a height, cross-section and a distance between one another that defines a space between the projections capable of generating capillary flow parallel to the substrate surface.

### CROSS REFERENCE TO RELATED APPLICATIONS

This patent application claims priority to Patent Cooperation Treaty Application Number PCT/US2013/022048 filed Jan. 18, 2013 and U.S. Provisional Application No. 61/588,779, filed Jan. 20, 2012, the disclosures of which are incorporated by reference in its entirety.

### FIELD OF THE INVENTION

The present invention relates to the field of diagnostic assays, and in particular to lateral flow assays where multiple analytes or controls are to be determined.

### BACKGROUND

Diagnostic assays are widespread and central for the diagnosis, treatment and management of many diseases. Different types of diagnostic assays have been developed over the years in order to simplify the detection of various analytes in clinical samples such as blood, serum, plasma, urine, saliva, tissue biopsies, stool, sputum, skin or throat swabs and tissue samples or processed tissue samples. These assays are frequently expected to give a fast and reliable result, while being easy to use and inexpensive to manufacture. Understandably, it is difficult to meet all these requirements in one and the same assay. In practice, many assays are limited by their speed. Another important parameter is sensitivity. Recent developments in assay technology have led to increasingly more sensitive tests that allow detection of an analyte in trace quantities as well the detection of disease indicators in a sample at the earliest time possible.

A common type of disposable assay device includes a zone or area for receiving the liquid sample, a conjugate zone also known as a reagent zone, and a reaction zone also known as a detection zone. These assay devices are commonly known as lateral flow test strips. They employ a porous material, e.g., nitrocellulose, defining a path for fluid flow capable of supporting capillary flow. Examples include those shown in U.S. Pat. Nos. 5,559,041, 5,714,389, 5,120,643, and 6,228,660 all of which are incorporated herein by reference in their entireties.

The sample-addition zone frequently consists of a more porous material, capable of absorbing the sample, and, when separation of blood cells is desired, also effective to trap the red blood cells. Examples of such materials are fibrous materials, such as paper, fleece, gel or tissue, comprising e.g. cellulose, wool, glass fiber, asbestos, synthetic fibers, polymers, or mixtures of the same.

Another type of assay device is a non-porous assay having projections to induce capillary flow. Examples of such assay devices include the open lateral flow device as disclosed in WO 2003/103835, WO 2005/089082, WO 2005/118139, and WO 2006/137785, all of which are incorporated herein by reference in their entireties.

A known non-porous assay device is shown in FIG. 1. The assay device 1, has at least one sample addition zone 2, a reagent zone 3, at least one detection zone 4, and at least one wicking zone 5. The zones form a flow path by which sample flows from the sample addition zone to the wicking zone. Also included are capture elements, such as antibodies, in the detection zone 4, capable of binding to the analyte, optionally deposited on the device (such as by coating); and a labeled conjugate material also capable of participating in reactions that will enable determination of the concentration of the analyte, deposited on the device in the reagent zone, wherein the labeled conjugate material carries a label for detection in the detection zone. The conjugate material is dissolved as the sample flows through the reagent zone forming a conjugate plume of dissolved labeled conjugate material and sample that flows downstream to the detection zone. As the conjugate plume flows into the detection zone, the conjugated material will be captured by the capture elements such as via a complex of conjugated material and analyte (as in a “sandwich” assay) or directly (as in a “competitive” assay. Unbound dissolved conjugate material will be swept past the detection zone into the at least one wicking zone 5.

An instrument such as that disclosed US 20060289787A1, US20070231883A1, U.S. Pat. No. 7,416,700 and U.S. Pat. No. 6,139,800 all incorporated by reference in their entireties, is able to detect the bound conjugated material in the detection zone. Common labels include fluorescent dyes that can be detected by instruments which excite the fluorescent dyes and incorporate a detector capable of detecting the fluorescent dyes.

The sample size for such typical assay devices as shown in FIG. 1 are generally on the order of 200 μl. Such a sample size requires a venous blood draw from a medical professional such as a phlebotomist. There is an increasing need for lateral flow devices that are able to function with a much smaller sample size to accommodate the amount of blood available from a so-called “fingerstick” blood draw, which is on the order of 25 μl or less. Such a small amount of sample is the amount of blood in a drop of blood after pricking a finger tip with a lancet. Home blood glucose meters typically use a drop of blood obtained in such a fashion to provide glucose levels in blood. Such a smaller sample size would not require a medical professional to draw the blood and would provide greater comfort to the patients providing the sample for analysis.

To reduce the sample size required, the dimensions of the lateral flow assay devices are reduced to accommodate the smaller sample size. However, it has been found that reducing the sample size and dimensions of the device, without more, provides inadequate conjugate in the detection zone and accordingly less signal that can be read by the instrument. The inadequate conjugate in the detection zone is believed to be due to reduced sample size and inefficient use of the sample in the device, amongst other conditions. Another drawback of reducing dimensions is that the width of the detection zone will also be reduced, again making less signal available that can be read by the instrument.

Another disadvantage with a typical assay design shown in FIG. 1 is that the length of the detection zone is very short and can only measure one analyte and cannot measure additional analytes or controls (e.g., internal positive and negative controls). While it is possible to increase the length of the detection zone along a straight line, this leads to an assay device that is larger than desired for point-of-care applications, has increased use of materials, and is more expensive to manufacture. In many instances, it would be desirable to have the ability to measure several analytes and controls, e.g., at least 6, or 8 or at least 10 analytes and/or controls.

The inventors conducted experiments using a devices that were modifications of the conventional size device as shown in FIG. 1. A list of the modified devices is shown in Table 1 below:

TABLE 1

| | Detection Zone | |
|---|---|---|
| Device Designs | Length | Max Analyte/Control Zones |
| A | 11 mm | 6 |
| B | 11 mm | 6 |
| C | 11 mm | 4 |
| D | 11 mm | 4 |
| E | 13.5 mm | 7 |
| F | 16 mm | 8 |

The modifications to the assay device are described below:
Design A and Design B (lengthened flow path by 7 mm over FIG. 1 design) without distinct reaction zones.
Design C and Design D (same flow channel length as Design B, but with 2 FIG. 1-like and 2 smaller (0.5×) distinct reaction zones).
Design E like Design B but with wicking zone volume reduced by 15% by reducing the wicking zone dimensions in the direction of flow and adding 2.5 mm of flow path length.
Design F like Design B but with the wicking zone volume reduced by 30% by reducing the wicking zone dimensions in the direction of flow and adding 5.0 mm of flow channel length compared to Design B.
The above modifications to the FIG. 1 device as described above did not allow 10 assays (detection zones) per device. The maximum number of detection zones that can be deposited for each of the modified devices is shown in Table 1 assuming that 2.0 mm center/center spacing is between each detection zone.

In addition, when the wicking zone was reduced in order to increase the length of the detection zone, such as shown by the Design F design, the wash volume was insufficient for the zone closest to the wicking zone. This is depicted in FIG. 2. Specifically, FIG. 2 shows, for each of the device modifications shown in Table 1, that the precision for the detection zone closest to the wicking zone is significantly worse than for other locations. For example, for modification Design B, the dimension of 31 mm designates the distance from the end of the device (i.e., this is furthest from the wicking zone). As shown for modification of Design F the imprecision as shown by the vertical line representing 2 standard deviations (SD) is significantly higher than the 2SD's for other locations.

FIG. 3A shows the imprecision observed for the Design F design when 10 detection zones spaced 1.5 mm were deposited on the Design F design. As the plot shows, the lack of precision is evident particularly at detection zones nearest the wicking zone as indicated by the overall lack of replicates and an increased level of imprecision (>25% CV). For both FIGS. 3A and 3B, the left side of the graph represents the detection zone closest to the wicking zone. FIG. 3B shows the precision attainable when 8 detection zones spaced 2 mm apart were deposited on the Design F design. As FIGS. 3A and 3B demonstrate, the precision attained for 8 zones was significantly better compared with 10 reaction zones. Finally FIG. 3C shows the coefficient of variation for each of the detection zones for both the 10 detection zones and 8 reaction zones on the Design F design.

Although the Design F design provided a satisfactory conventionally sized device for having 8 detection zones, there was still a need for a device that could be capable of supporting greater than 8 detection zones and that can use a smaller sample size, such as a sample size on the order of 25 μl as described above and a smaller footprint (i.e., smaller dimensions).

Accordingly, there is a need for an assay device that can provide a multiple detection zones in a small footprint using less sample, while maintaining precision of the results, while at the same time providing more detection zones.

### SUMMARY OF THE INVENTION

The present invention is directed to an assay device that alleviates one or more the foregoing problems described above.

One aspect of the invention is directed to an assay device for determining the concentration of multiple analytes or controls where the device is capable of determining the presence or concentration of at least six analytes or controls that includes: a fluid flow path; a liquid sample addition zone; a reagent zone downstream and in fluid communication with the sample addition zone containing one or more reagents; multiple detection zones in fluid communication with the reagent zone, wherein the fluid flow path which extends through the detection zones has a length capable of having at least six detection zones linearly spaced at least a sufficient distance apart in order to discriminate each signal peak from its adjacent signal peak; and a wicking zone in fluid communication with the detection zones having a capacity to receive liquid sample flowing from the detection zone, wherein the fluid flow path extends from the sample zone to the wicking zone, and at least a part of the fluid flow path has a substrate and projections which extend substantially vertically from the substrate, wherein the projections have a height, cross-section and a distance between one another that defines a space between the projections capable of generating capillary flow parallel to the substrate surface.

According to another aspect of the invention, there has been provided a method of performing an assay on a liquid sample for the presence or concentration of multiple analytes or controls, on an assay device capable of determining the presence or concentration of at least six analytes or controls that includes: depositing a liquid sample containing the analyte(s) of interest onto a sample addition zone of an assay device; moving the sample by capillary action through a fluid flow path into a reagent zone where it dissolves one or more reagents; flowing the sample away from the reagent zone having a dissolved reagent plume containing one or more reagents and into multiple detection zones by capillary action through the fluid flow path, wherein the fluid flow path which extends through detection zones has a length capable of having at least six zones spaced at least a sufficient distance apart in order to discriminate each signal peak from its adjacent signal peak, wherein signals representative of the presence or concentration of analyte(s) or control(s) is produced; reading the signals that are produced in the multiple detection zones to determine the presence or concentration of the multiple analytes or controls.

According to yet another aspect of the invention, there has been provided a method for determining the concentration of multiple analytes or controls where the device is capable of determining the presence or concentration of at least six analytes or controls that includes: a fluid flow path; a liquid sample addition zone; a reagent zone downstream and in fluid communication with the sample addition zone containing one or more reagents; multiple detection zones in fluid communication with the reagent zone, wherein the fluid flow path which extends through the detection zones is split into multiple parallel flow paths each flow path having a detection zone therein, wherein the detection zones are placed at least a sufficient distance apart in order to discriminate each signal peak from its adjacent signal peak; and a wicking zone in fluid communication with the detection zones having a capacity to receive liquid sample flowing from the detection zone, wherein the fluid flow path extends from the sample zone to the wicking zone, and at least a part of the fluid flow path has a substrate and projections which extend substantially vertically from the substrate, wherein the projections have a height, cross-section and a distance between one another that defines a space between the projections capable of generating capillary flow parallel to the substrate surface.

Further objects, features and advantages of the present invention will be apparent to those skilled in the art from detailed consideration of the preferred embodiments that follow.

### BRIEF DESCRIPTION OF THE DRAWINGS

FIG. 1 shows a known assay device.

FIG. 2 shows experimental data showing the poor precision attained for the detection zone nearest the wicking zone for a conventional assay device having a lengthened flow path and a smaller wicking zone.

FIGS. 3A and 3B show experimental data demonstrating the superior precision attained for a conventionally sized assay device having detections zones 2 mm apart vs. 1.5 mm apart. Individual traces denote individual replicates of the same sample.

FIG. 4 the coefficient of variation for the two assay devices depicted in FIGS. 3A and 3B.

FIG. 5A shows a schematic view of an assay device according to one embodiment of the invention having 10 detection zones.

FIG. 5B shows a schematic view of an assay device according to one embodiment of the invention capable of supporting 10 detection zones.

FIG. 6 shows a schematic view of an assay device according to one embodiment of the invention capable of supporting 10 detection zones.

FIG. 7A shows a dose response curve for CRBM at the second detection zone closest to the wicking zone, which is the second peak from the left in FIG. 7B, while holding the concentrations of NT-proBNP and PHBR constant.

FIG. 7B shows experimental results demonstrating the ability to conduct both multiple immunometric (i.e., sandwich-type) assays and multiple competitive binding assays on the same assay device. The three traces have different concentrations of CRBM.

FIGS. 8A-C shows experimental dose response curves for NT-proBNP, PIGF, and iPTH, respectively.

FIG. 9 shows experimental results demonstrating the ability to perform 10 competitive assays on an assay device according to the present invention.

FIGS. 10A and 10B show dose response curves for Ariprazole.

FIGS. 11A and 11B show dose response curves for Olanzapine.

FIGS. 12A and 12B show dose response curves for Quetiapine.

FIGS. 13A and 13B show dose response curves for Risperidone.

### DETAILED DESCRIPTION OF PREFERRED EMBODIMENTS

As used in this specification and the appended claims, the singular forms “a”, “an” and “the” include plural referents unless the context clearly dictates otherwise.

The term “about” as used in connection with a numerical value throughout the description and the claims denotes an interval of accuracy, familiar and acceptable to a person skilled in the art. The interval is preferably ±10%.

The term “sample” herein means a volume of a liquid, solution or suspension, intended to be subjected to qualitative or quantitative determination of any of its properties, such as the presence or absence of a component, the concentration of a component, etc. Typical samples in the context of the present invention are human or animal bodily fluids such as blood, plasma, serum, lymph, urine, saliva, semen, amniotic fluid, gastric fluid, phlegm, sputum, mucus, tears, stool, etc. Other types of samples are derived from human or animal tissue samples where the tissue sample has been processed into a liquid, solution, or suspension to reveal particular tissue components for examination. The embodiments of the present invention are applicable to all bodily samples, but preferably to samples of whole blood, urine or sputum.

In other instances, the sample can be related to food testing, environmental testing, bio-threat or bio-hazard testing, etc. This is only a small example of samples that can be used in the present invention.

In the present invention, the determination based on lateral flow of a sample and the interaction of components present in the sample with reagents present in the device or added to the device during the procedure and detection of such interaction, either qualitatively or quantitatively, may be for any purpose, such as diagnostic purposes. Such tests are often referred to as lateral flow assays.

Examples of diagnostic determinations include, but are not limited to, the determination of analytes, also called markers, specific for different disorders, e.g. chronic metabolic disorders, such as blood glucose, blood ketones, urine glucose (diabetes), blood cholesterol (atherosclerosis, obesity, etc); markers of other specific diseases, e.g. acute diseases, such as coronary infarct markers (e.g. troponin-T, NT-ProBNP), markers of thyroid function (e.g. determination of thyroid stimulating hormone (TSH)), markers of viral infections (the use of lateral flow immunoassays for the detection of specific viral antibodies); etc.

Yet another important field is the field of companion diagnostics where a therapeutic agent, such as a drug, is administered to an individual in need of such a drug. An appropriate assay is then conducted to determine the level of an appropriate marker to determine whether the drug is having its desired effect. Alternatively, the assay device of the present invention can be used prior to administration of a therapeutic agent to determine if the agent will help the individual in need.

Yet another important field is that of drug tests, for easy and rapid detection of drugs and drug metabolites indicating drug abuse; such as the determination of specific drugs and drug metabolites (e.g. THC) in urine samples etc.

The term “analyte” is used as a synonym of the term “marker” and intended to encompass any chemical or biological substance that is measured quantitatively or qualitatively and can include small molecules, proteins, antibodies, DNA, RNA, nucleic acids, virus components or intact viruses, bacteria components or intact bacteria, cellular components or intact cells and complexes and derivatives thereof.

The terms “zone”, “area” and “site” are used in the context of this description, examples and claims to define parts of the fluid flow path on a substrate, either in prior art devices or in a device according to an embodiment of the invention.

The term “reaction” is used to define any reaction, which takes place between components of a sample and at least one reagent or reagents on or in the substrate, or between two or more components present in the sample. The term “reaction” is in particular used to define the reaction, taking place between an analyte and a reagent as part of the qualitative or quantitative determination of the analyte.

The term “substrate” means the carrier or matrix to which a sample is added, and on or in which the determination is performed, or where the reaction between analyte and reagent takes place.

The present invention is directed to a lateral flow assay device for determining the presence or amount of multiple analytes or controls that solves, at least in part, the problem of insufficient length in the flow path to enable at least six assays or controls and reduced sample size to meet the requirement of fingerstick volumes of blood. Such small volumes make it difficult to provide sufficient amount of liquid to dissolve the reagent material (described below) and to provide sufficient amount of wash to wash sample and any other materials into the wicking zone, which in turn, affects the precision of at least some of the multiple detections zones, particularly those closest to the wicking zone.

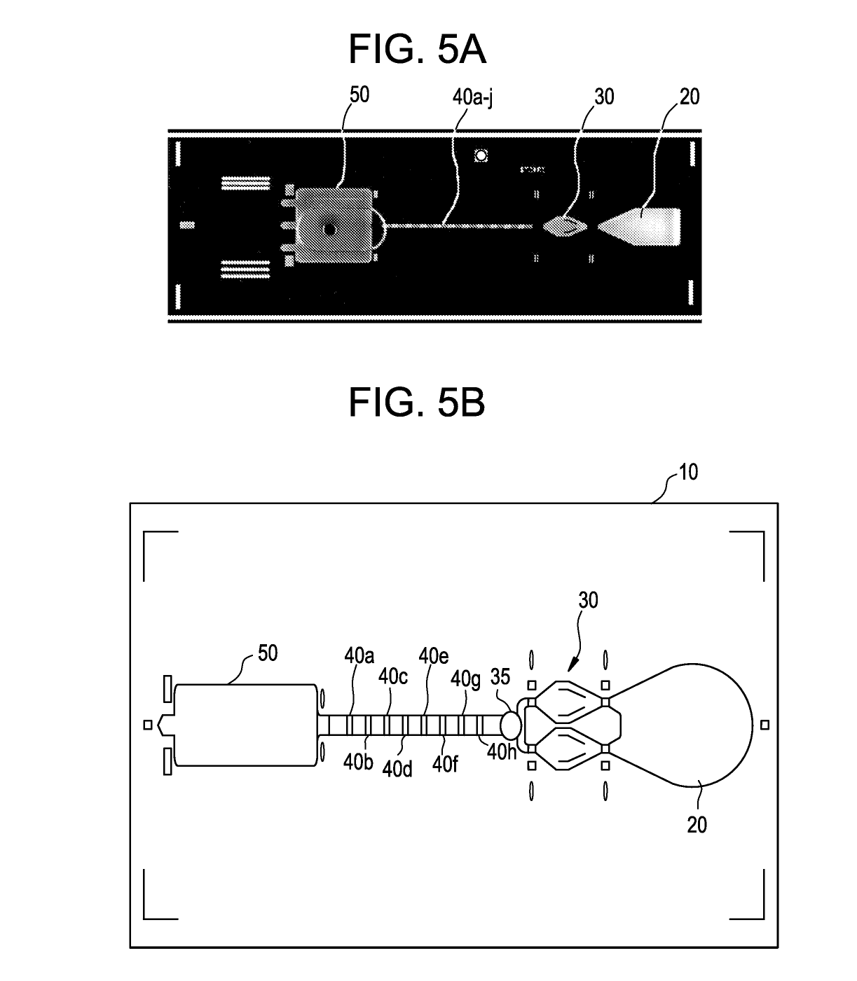

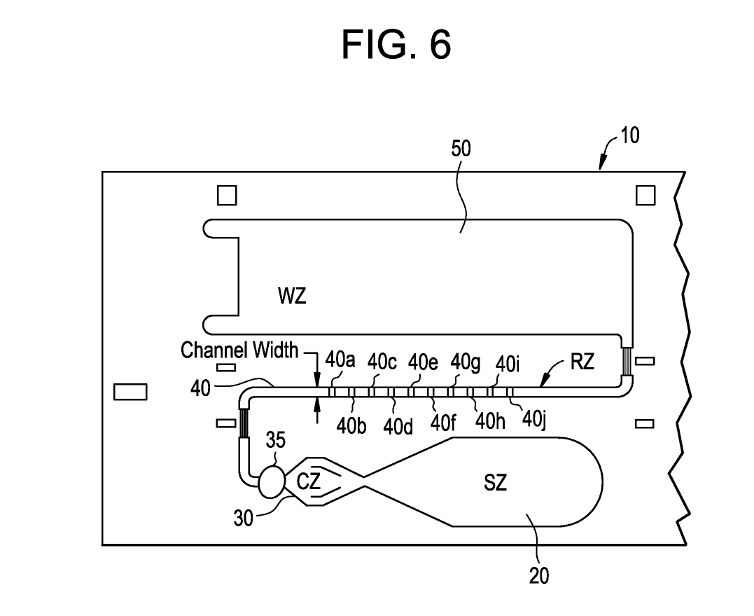

FIGS. 5A, 5B and 6 show schematic views of preferred embodiments of such a device according to the invention. The assay device 10, has at least one sample addition zone 20, at least one reagent zone 30, multiple detection zones 40a, 40b, 40c, etc., with the device capable of having at least 6 detection zones, and at least one wicking zone 50. The detection zone are described more fully below. The zones form a flow path by which sample flows from the sample addition zone to the wicking zone.

Components of the assay device (i.e., a physical structure of the device whether or not a discrete piece from other parts of the device) can be prepared from copolymers, blends, laminates, metalized foils, metalized films or metals. Alternatively, device components can be prepared from copolymers, blends, laminates, metalized foils, metalized films or metals deposited one of the following materials: polyolefins, polyesters, styrene containing polymers, polycarbonate, acrylic polymers, chlorine containing polymers, acetal homopolymers and copolymers, cellulosics and their esters, cellulose nitrate, fluorine containing polymers, polyamides, polyimides, polymethylmethacrylates, sulfur containing polymers, polyurethanes, silicon containing polymers, glass, and ceramic materials. Alternatively, components of the device are made with a plastic, elastomer, latex, silicon chip, or metal; the elastomer can comprise polyethylene, polypropylene, polystyrene, polyacrylates, silicon elastomers, or latex. Alternatively, components of the device can be prepared from latex, polystyrene latex or hydrophobic polymers; the hydrophobic polymer can comprise polypropylene, polyethylene, or polyester. Alternatively, components of the device can comprise TEFLON®, polystyrene, polyacrylate, or polycarbonate. Alternatively, device components are made from plastics which are capable of being embossed, milled or injection molded or from surfaces of copper, silver and gold films upon which may be adsorbed various long chain alkanethiols. The structures of plastic which are capable of being milled or injection molded can comprise a polystyrene, a polycarbonate, or a polyacrylate. In a particularly preferred embodiment, the assay device is injection molded from a cyclo olefin polymer, such as those sold under the name Zeonor®. Preferred injection molding techniques are described in U.S. Pat. Nos. 6,372,542, 6,733,682, 6,811,736, 6,884,370, and 6,733,682, all of which are incorporated herein by reference in their entireties.

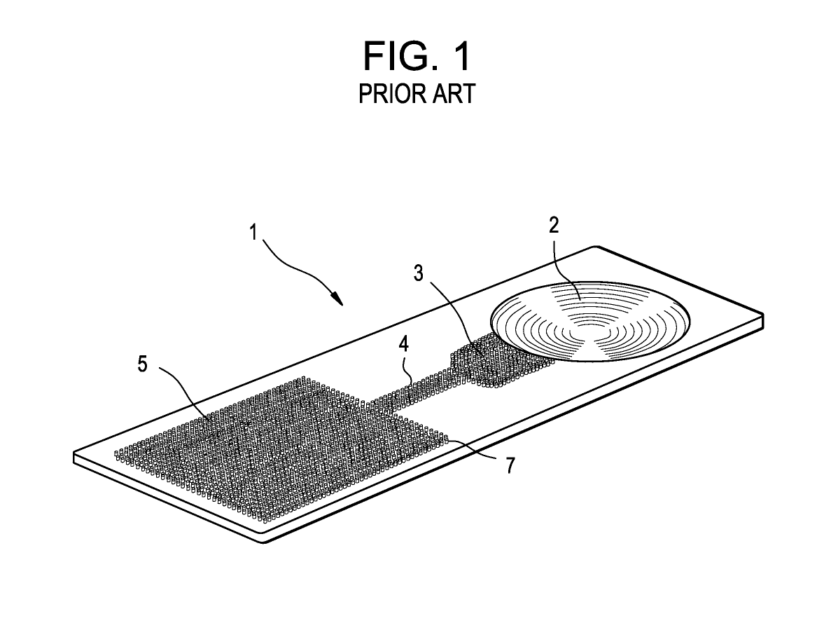

The flow path can include open or closed paths, grooves, and capillaries. Preferably the flow path comprises a lateral flow path of adjacent projections, having a size, shape and mutual spacing such that capillary flow is sustained through the flow path. In one embodiment, the flow path is in a channel within the substrate having a bottom surface and side walls. In this embodiment, the projections protrude from the bottom surface of the channel. The side walls may or may not contribute to the capillary action of the liquid. If the sidewalls do not contribute to the capillary action of the liquid, then a gap can be provided between the outermost projections and the sidewalls to keep the liquid contained in the flow path defined by the projections. FIG. 1 shows projections 7.

In one embodiment the flow path is at least partially open. In another embodiment the flow path is entirely open. Open means that there is no lid or cover at a capillary distance. Thus the lid, if present as a physical protection for the flow path, does not contribute to the capillary flow in the flow path. An open lateral flow path is described for example in the following published applications: WO 2003/103835, WO 2005/089082; WO 2005/118139; WO 2006/137785; and WO 2007/149042, all of which are incorporated by reference in their entireties. The projections have a height (H), diameter (D) and a distance or distances between the projections (t1, t2) such, that lateral capillary flow of the fluid, such as plasma, preferably human plasma, in the zone is achieved. These dimensions are shown in US 2006/0285996, which is incorporated by reference in its entirety. In addition to optimizing the above-mentioned height, diameter and a distance or distances between the projections, the projections may be given a desired chemical, biological or physical functionality, e.g. by modifying the surface of the projections. In one embodiment, the projections have a height in the interval of about 15 to about 150 μm, preferably about 30 to about 100 μm, a diameter of about 10 to about 160 μm, preferably 40 to about 100 μm, and a gap or gaps between the projections of about 3 to about 200 μm, preferably 5 to about 50 μm or 10 to 50 μm from each other. The flow channel may have a length of about 5 to about 500 mm, preferably about 10 to about 100 mm, and a width of about 0.3 to about 10 mm, preferably about 0.3 to about 3 mm.

While most detection will occur in the detection zone portion of the fluid flow path, it is also possible that detection may occur in other parts of the device. For example, non-invasive, non-reactive sample integrity measurements may occur between the sample zone and the reagent zone or reagent addition zone, preferably after a filter element, if present. Other measurements may include blank reads, one part of a two part reaction sequence as for measuring both hemoglobin and glycated hemoglobin for determination of HbA1c, etc.

The liquid sample zone 20, also referred to as the liquid sample addition zone, receives sample from a sample dispenser, such as a pipette. The sample is typically deposited onto the top of the zone. The sample addition zone is capable of transporting the liquid sample from the point where the sample is deposited to the reagent zone, through an optional filter and reagent addition zone, preferably through capillary flow. The capillary flow inducing structure can include porous materials, such as nitrocellulose, or preferably through projections, such as micro-pillars, as shown in FIG. 1. In those devices that can use finger stick volumes of blood, the sample can be directly touched off from the finger, or by a capillary pipette such as described in co pending application entitled “Controlling Fluid Flow Through An Assay Device” (Application No. 61/588,772, Attorney Docket No. CDS5112USPSP, first named inventor: James Kanaley), filed Jan. 20, 2012 are incorporated by reference in its entirety.

A filter material (not shown) can be placed in the sample addition zone to filter particulates from the sample or to filter blood cells from blood so that plasma can travel further through the device.

Located between the sample addition zone and the detection zone is a reagent zone 30. The reagent zone can include reagent(s) integrated into the analytical element and are generally reagents useful in the reaction—binding partners such as antibodies or antigens for immunoassays, substrates for enzyme assays, probes for molecular diagnostic assays, or are auxiliary materials such as materials that stabilize the integrated reagents, materials that suppress interfering reactions, etc. Generally one of the reagents useful in the reaction bears a detectable signal as discussed below. In some cases the reagents may react with the analyte directly or through a cascade of reactions to form a detectable signal such as, but not restricted to, a molecule detectable using spectroscopy such as a colored or fluorescent molecule. The amount of reagent in the reagent zone can be adjusted by the length of reagent deposited into the device while maintaining the same reagent width. The amount of reagent can also be adjusted by changing the width while maintaining the length. The amount of reagent can further be adjusted by changing both width and length simultaneously. In one preferred embodiment, the reaction zone includes conjugate material. The term conjugate means any moiety bearing both a detection element and a binding partner.

The detection element is an agent which is detectable with respect to its physical distribution or/and the intensity of the signal it delivers, such as but not limited to luminescent molecules (e.g. fluorescent agents, phosphorescent agents, chemiluminescent agents, bioluminescent agents and the like), colored molecules, molecules producing colors upon reaction, enzymes, radioisotopes, ligands exhibiting specific binding and the like. The detection element, also referred to as a label, is preferably chosen from chromophores, fluorophores, radioactive labels, and enzymes. Suitable labels are available from commercial suppliers, providing a wide range of dyes for the labeling of antibodies, proteins, and nucleic acids. There are, for example, fluorophores spanning practically the entire visible and infrared spectrum. Suitable fluorescent or phosphorescent labels include for instance, but are not limited to, fluoresceins, Cy3, Cy5 and the like. Suitable chemoluminescent labels are for instance but are not limited to luminol, cyalume and the like.

Similarly, radioactive labels are commercially available, or detection elements can be synthesized so that they incorporate a radioactive label. Suitable radioactive labels are for instance but are not limited to radioactive iodine and phosphorus; e.g. ¹²⁵I and ³²P.

Suitable enzymatic labels are, for instance, but are not limited to, horseradish peroxidase, beta-galactosidase, luciferase, alkaline phosphatase and the like. Two labels are “distinguishable” when they can be individually detected and preferably quantified simultaneously, without significantly disturbing, interfering or quenching each other. Two or more labels may be used, for example, when multiple analytes or markers are being detected.

The binding partner is a material that can form a complex that can be used to determine the presence of or amount of an analyte. For example, in an “sandwich” assay, the binding partner in the conjugate can form a complex including the analyte and the conjugate and that complex can further bind to another binding partner, also called a capture element, integrated into the detection zone. In a competitive immunoassay, the analyte will interfere with binding of the binding partner in the conjugate to another binding partner, also called a capture element, integrated into the detection zone. Example binding partners included in conjugates include antibodies, antigens, analyte or analyte-mimics, protein, etc.

Optionally located in the fluid flow path, before or after the reagent zone and before the detection zone is a reagent addition zone. The reagent addition zone is shown as 35 in FIGS. 5A, 5B and 6. The reagent addition zone can allow addition of a reagent externally from the device. For example, the reagent addition zone may be used to add an interrupting reagent that may be used to wash the sample and other unbound components present in the fluid flow path into the wicking zone. In a preferred embodiment the reagent addition zone 35 is located after the reagent zone 30.

Downstream from the liquid sample addition zone are the multiple detection zones 40a, 40b, 40c, etc., shown in FIGS. 5A-B and 6, which are in fluid communication with the sample addition zone. The detection zones 40 may include projections such as those described above. As also noted above, these projections are preferably integrally molded into the substrate from an optical plastic material such as Zeonor, such as injection molding or embossing. The width of the flow channel in the detection zone is typically on the order of 2 mm for conventional size devices, however, some lower volume devices, such as those described above and in copending application, entitled “Low Volume Assay Device Having Increased Sensitivity” (Application No. 61/588,758, Attorney Docket No. CDS 5111USPSP, first named inventor: Phil Hosimer) filed Jan. 20, 2012 and incorporated by reference in its entirety, are significantly narrower, e.g., 1.5 mm or less.

The detection zone is where any detectable signal is read. In a preferred embodiment attached to the projections in the detection zone are capture elements. The capture elements can include binding partners for the conjugate or complexes containing the conjugate, as described above. For example, if the analyte is a specific protein, the conjugate may be an antibody that will specifically bind that protein coupled to a detection element such as a fluorescence probe. The capture element could then be another antibody that also specifically binds to that protein. In another example, if the marker or analyte is DNA, the capture molecule can be, but is not limited to, synthetic oligonucleotides, analogues thereof, or specific antibodies. Other suitable capture elements include antibodies, antibody fragments, aptamers, and nucleic acid sequences, specific for the analyte to be detected. A non-limiting example of a suitable capture element is a molecule that bears avidin functionality that would bind to a conjugate containing a biotin functionality. As noted above, the detection zone can include multiple detection zones. The multiple detection zones can be used for assays that include one or more markers. In the event of multiple detection zones, the capture elements can include multiple capture elements, such as first and second capture elements. The conjugate can be pre-deposited on the assay device, such as by coating in the reagent zone. Similarly the capture elements can be pre-deposited on the assay device on the detection zone. Preferably, both the detection and capture elements are pre-deposited on the assay device, on the reaction zone and detection zone, respectively.

After the sample has been delivered to the sample zone, it will encounter the reagent zone. After the sample has flowed through and interacted with the reagent zone and optionally the reagent addition zone, the sample and a reagent plume will be contained in the fluid flow. The reagent plume can contain any of the reagent materials that have been dissolved in the reaction zone or those added through the reagent addition zone. The reagent plume can include the conjugate having both the detection element and binding partner, in which case it is often referred to as a conjugate plume. As noted throughout, one challenge facing the inventors was to keep the reagent plume as wide as possible as it enters the detection zone.

The present invention is based, in part, on the surprising discovery that reducing the size of the assay device and the resulting volume of sample actually made it possible to have more assays and/or controls (i.e., more detection zones) than on a conventionally sized device such as shown in FIG. 1. This was particularly surprising because the skilled artisan would have thought a larger sample size and a larger size device was need to conduct more assays or controls.

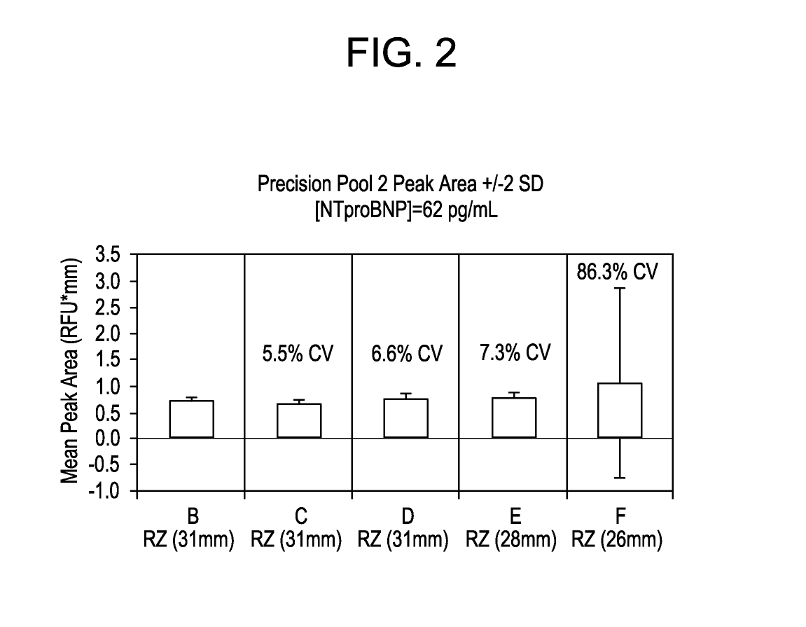

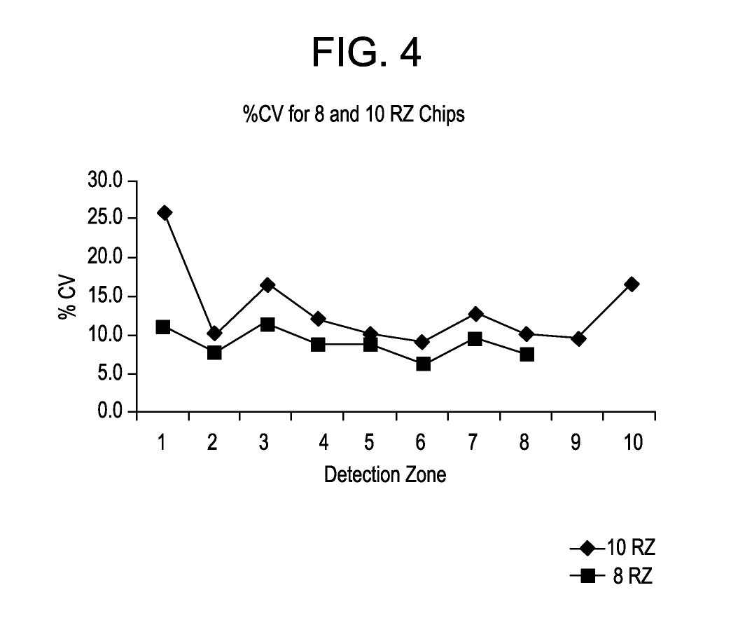

The present inventors discovered modifying an assay device such as described in copending applications entitled “Low Volume Assay Device Having Increased Sensitivity” (Application No. 61/588,758, Attorney Docket No. CDS 5111 USPSP, first named inventor: Phil Hosimer), “Assay Device Having Multiple Reagent Cells” (Ser. No. 61/588,738, Attorney Docket No. CDS5104USPSP, first named inventor Zhong Ding), “Assay Device Having Uniform Flow Around Corners” (Application No. 61/588,745, Attorney Docket No. CDS5110USPSP, first named inventor James Kanaley), “Controlling Fluid Flow Through An Assay Device” (Application No. 61/588,772, Attorney Docket No. CDS5112USPSP, first named inventor James Kanaley), and “Assay Device Having Controllable Sample Size” (Application No. 61/588,899, Attorney Docket No. CDS5114USPSP, first named inventor, Ed Scalice), all filed Jan. 20, 2012 and all incorporated by reference in their entireties, provided a platform where multiplexing could be achieved. Briefly, the inventors discovered that providing corners in the flow path while maintaining uniform flow of fluid through those corners, allowed the flow path containing the detection zones to be increased, e.g., up to 20 mm or more, while still maintaining a smaller size device and smaller sample size. This increased flow path length provides the needed dimensions for the multiple detection zones, e.g., at least six, more preferably at least 8 and most preferably at least 10 detection zones. The inventors also discovered that providing a narrower width in the flow path containing the detection zones. e.g. on the order of 0.5 to 1.5 mm wide, more preferably 0.5 to 1.2 mm wide and a wider reagent plume from the reagent zone or reagent addition zone, also contributes to a greater signal being read and hence a need for less sample. At the same time, reducing sample size surprisingly allows the ratio of wash/sample to be increased, so the detection zones nearest the wicking zone will not suffer from the lack of precision as shown in FIGS. 2-4.

In addition to providing the ability to have multiple detection zones (i.e., also known as multiplexing) on a single assay device, the improved assay device made possible by the discoveries described in the above-mentioned copending applications also make it possible to have different types of assay formats in multiple locations on a single device. For example, different assay formats can include heterogeneous assays (i.e., sandwich-type) and competitive assays, etc.

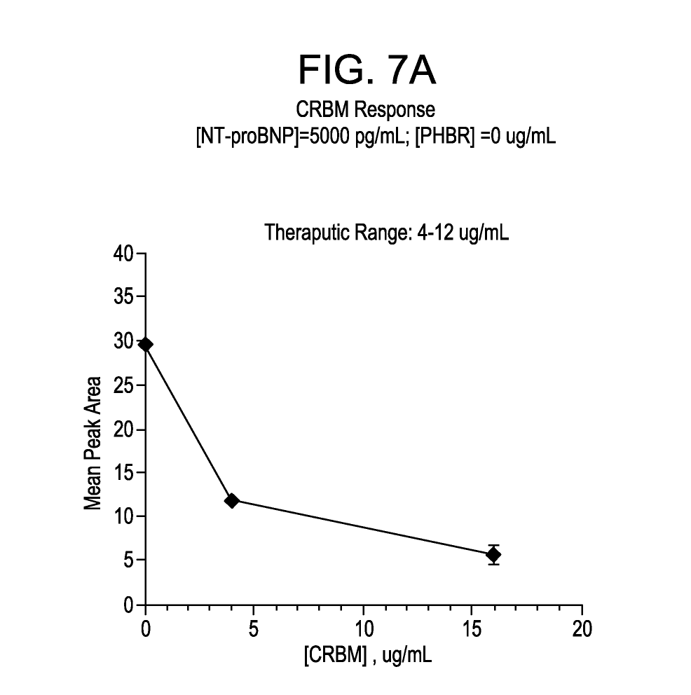

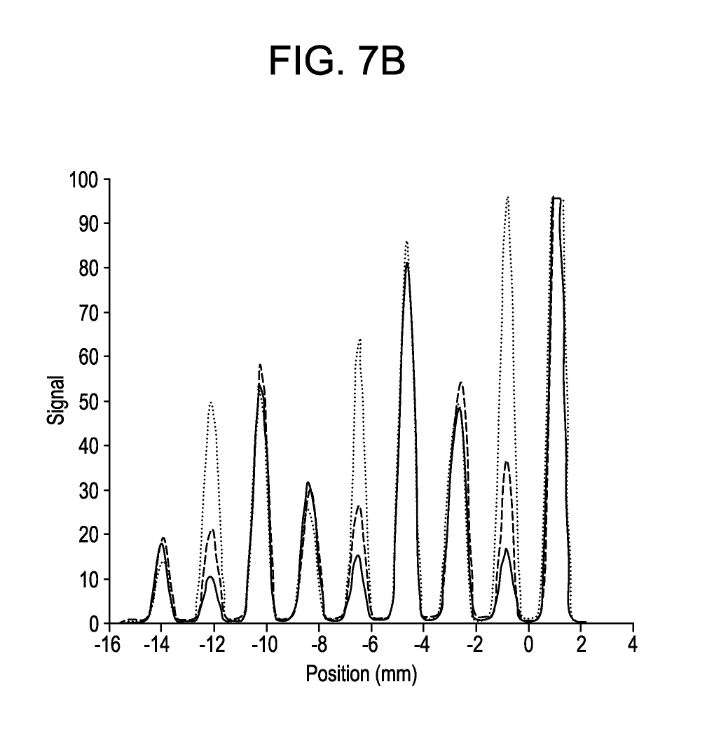

FIGS. 7A and 7B demonstrate the ability to conduct 2 different assay formats using three different analytes for a total of 9 detection zones. The x-axis of FIG. 7B shows the position of the detection zone relative to the wicking zone, with the left hand side of the x-axis being closest to the wicking zone. The y-axis of FIG. 7B shows the signal strength as measure in peak area (rfu). Specifically, NT-proBNP (BNP) a sandwich-type assay, carbamazepine (CRBM) and Phenobarbital (PHBR), both competitive assays, were each deposited in three different locations in the fluid flow path for a total of 9 detection zones. The position of each of the analytes is shown along the top of FIG. 7B. For the experiments shown in FIGS. 7A and 7B, three different runs were performed. For each run, the concentrations of BNP and PHBR were held constant, whereas the concentration of CRBM varied. Hence the reasons for three distinct peaks in the CRBM positions in FIG. 7B. The device had a 0.5 mm flow path width in the detection zones, and a single conjugate zone as shown in FIG. 5B. As FIG. 7B shows, each of the 9 positions demonstrate a clear signal that is distinguishable from its neighboring signals. As expected, the detection zones nearest the wicking zone for each of the assays had the lowest signal strength due to depletion of the analyte as it moves away from the sample addition zone to the wicking zone, but still had good precision. As also expected for the competitive assays, the signal strength increases at lower concentrations of analyte in the sample. FIG. 7A shows a dose response curve of CRBM at the second detection zone (closest to the wicking zone) from FIG. 7B. FIG. 7 B demonstrates that the presence of BNP and PHBR does not significantly affect the signal of CRBM.

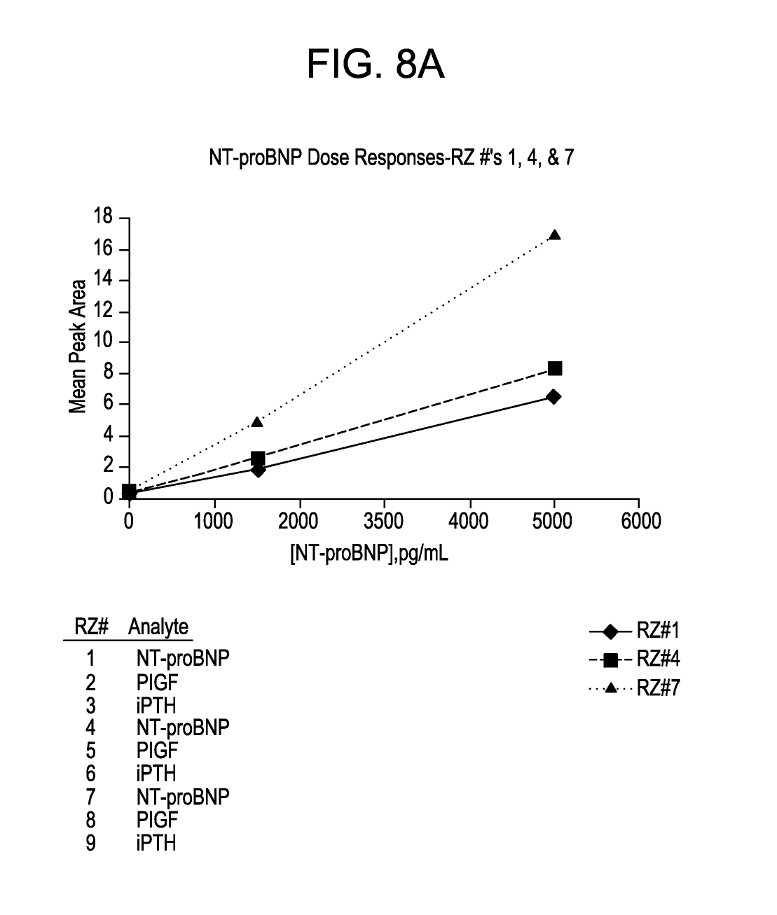

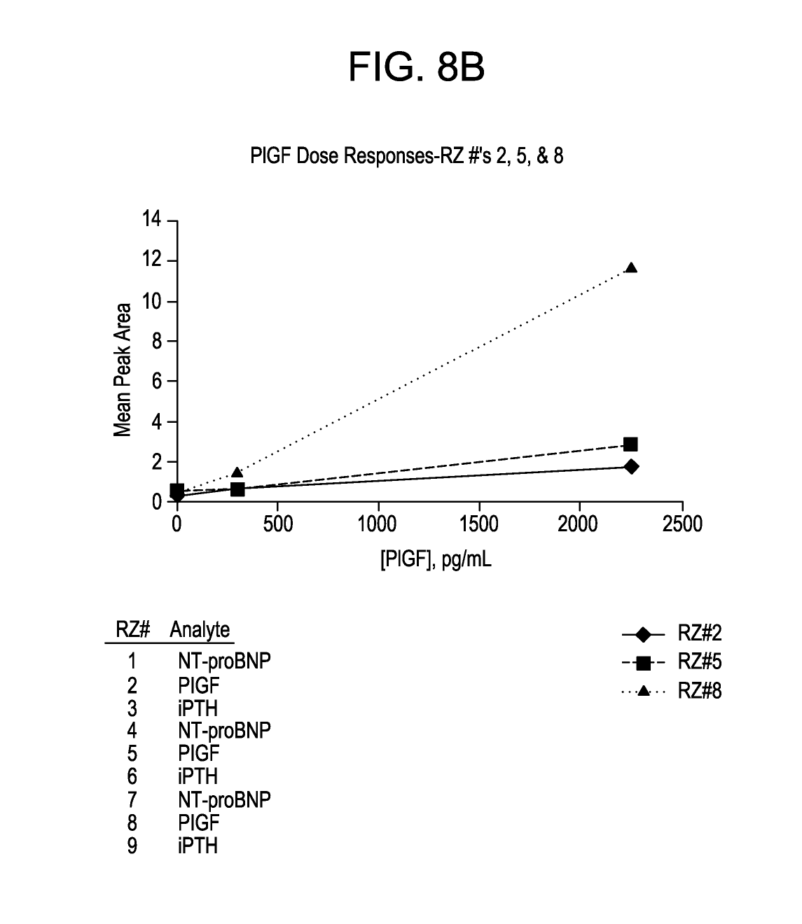

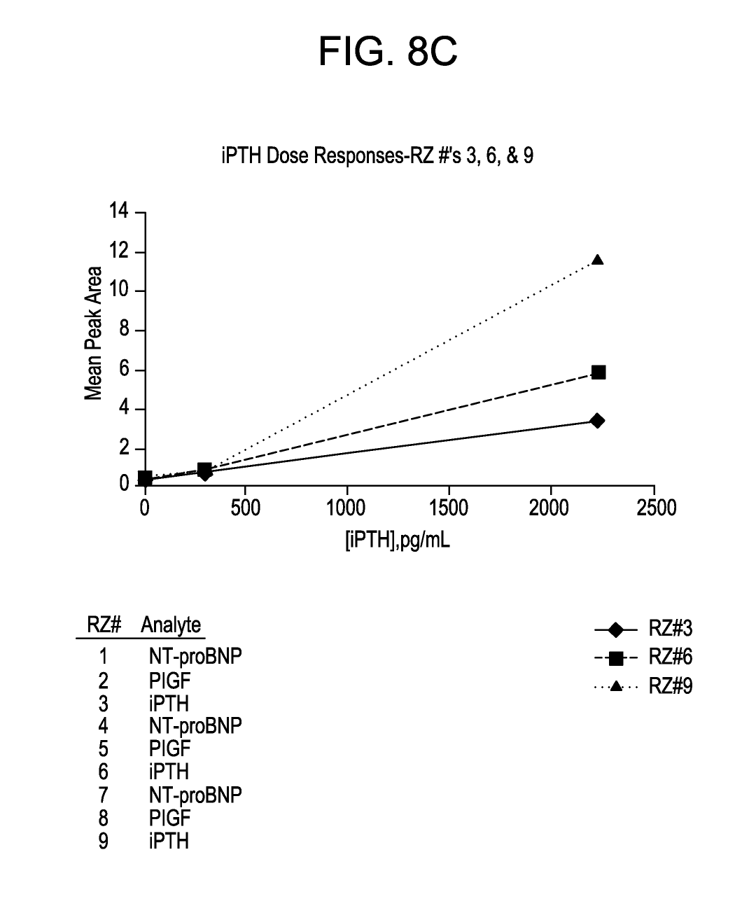

FIGS. 8A-8C shows the ability to conduct 9 immunometric (i.e., sandwich-type) assays on a single device. Three different assays types (NTproBNP, PIGF, and iPTH) were each deposited at 3 different detection zones for a total of 9 assays. FIG. 8A shows a dose response curve for NT-proBNP deposited at three different detection zones represented by RZ#1 (diamond), RZ#4 (square) and RZ#7 (triangle). RZ#1 is the detection zone nearest the wicking zone, RZ#2 is the next closest position and so on up through RZ#9 which is furthest from the wicking zone. The x-axis in all FIGS. 8A-8C represents the concentration of the analyte in pg/ml and the y-axis represents the signal strength as represented by peak area (rfu). FIG. 8A shows that for each position along the assay device, the dose response curve is acceptable. As described above, less signal is produced farther from the sample zone due to depletion of the analyte. Hence, the reason that the curve for positions RZ#1 and RZ#4 is flatter than the curve at RZ#7.

Likewise, 8B shows a dose response curve for PIGF deposited at three different detection zones represented by RZ#2 (diamond), RZ#5 (square) and RZ#8 (triangle). FIG. 8B shows that for each position along the assay device, the dose response curve is acceptable. As described above, less signal is produced farther from the sample zone due to depletion of the analyte. Hence, the reason that the curve for positions RZ#5 and RZ#2 is flatter than the curve at RZ#8.

Likewise, 8C shows a dose response curve for iPTH deposited at three different detection zones represented by RZ#3 (diamond), RZ#6 (square) and RZ#9 (triangle). FIG. 8C shows that for each position along the assay device, the dose response curve is acceptable. As described above, less signal is produced farther from the sample zone due to depletion of the analyte. Hence, the reason that the curve for positions RZ#5 and RZ#2 is flatter than the curve at RZ#8.

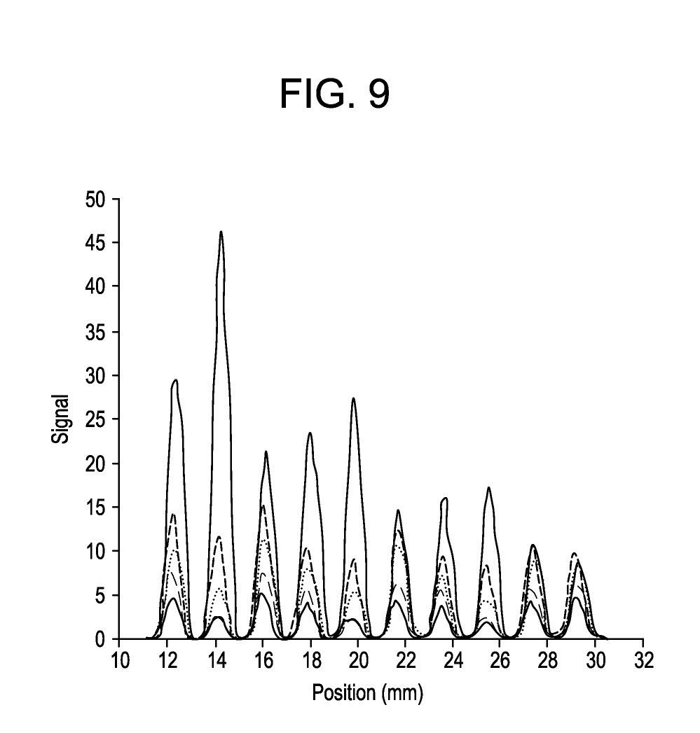

FIG. 9 demonstrates the ability to multiplex 10 different competitive assays on an assay device such as that shown in FIG. 7 with the flow path having a width of 0.5 mm through the multiple detection zones spaced 2 mm apart and a having a total length of 20 mm. Three competitive assays CRBM, PHBR and risperidone (RISP) were each deposited multiple times along the fluid flow paths at different lengths from the wicking zone. In FIG. 9, the y-axis shows the position of the detection zone represented by RZ#1, RZ#2 . . . RZ#10) relative to the wicking zone with RZ#10 being farthest from the wicking zone and RZ #1 being closest to the wicking zone. The signal (rfu) is shown on the y-axis. At each position or detection zone a representative curve is shown for 5 different concentrations of none (no analyte present), low, mid, mid-high and high concentrations of analyte. As FIG. 9 shows that for each position at differing concentrations, a clear signal that is distinguishable from the signals in adjacent detection zones is obtained. In other words, the assay device allows the detection zones to be spaced at least a sufficient distance apart in order to discriminate each signal peak from its adjacent signal peak.

Downstream from the detection zone is a wicking zone in fluid communication with the detection zone. The wicking zone is an area of the assay device with the capacity of receiving liquid sample and any other material in the flow path, e.g., unbound reagents, wash fluids, etc. The wicking zone provides a capillary force to continue moving the liquid sample through and out of the detection zone. The wicking zone can include a porous material such as nitrocellulose or can be a non-porous structure such as the projections described herein. The wicking zone can also include non-capillary fluid driving means, such as using evaporative heating or a pump. Further details of wicking zones as used in assay devices according to the present invention can be found in patent publications US 2005/0042766 and US 2006/0239859, both of which are incorporated herein by reference in their entireties. Wicking zones are also described in copending patent application entitled “Controlling Fluid Flow Through An Assay Device” (Application No. 61/588,772, Attorney Docket No. CDS 5112USPSP, first named inventor: James Kanaley), filed Jan. 20, 2012 and incorporated by reference in its entirety.

Preferably the entirety of the flow path including the sample addition zone, the detection zone and the wicking zone includes projections substantially vertical in relation to the substrate, and having a height, diameter and reciprocal spacing capable of creating lateral flow of the sample in the flow path.

In any of the above embodiments, the device is preferably a disposable assay device. The assay device may be contained in a housing for ease of handling and protection. If the assay device is contained in such a housing, the housing will preferably include a port for adding sample to the assay device.

The assay device of the present invention can be used with a device for reading (a reader) the result of an assay device performed on the assay of the present invention. The reader includes means for reading a signal emitted by, or reflected from the detection element, such as a photodetector, and means for computing the signal and displaying a result, such as microprocessor that may be included within an integrated reader or on a separate computer. Suitable readers are described for example in US 2007/0231883 and U.S. Pat. No. 7,416,700, both of which are incorporated by reference in their entireties.

Another embodiment is a device for reading the result of an assay performed on an assay device, wherein the device comprises a detector capable of reading a signal emitted from or reflected from at least one detection element present in a defined location of the assay device. In either of the above embodiments, the reading preferably is chosen from the detection and/or quantification of color, fluorescence, radioactivity or enzymatic activity.

Another aspect of the invention is directed to a method of performing an assay on a liquid sample for detecting the presence or concentration of multiple analytes of interest or controls. As noted above, the assay device has been constructed in such a manner according to the present invention that it is capable of determining the presence or concentration of at least six analytes or controls. A liquid sample containing the analyte(s) of interest is deposited onto the sample zone of the assay device, such as through a port in the housing of the device, or by touching off a finger directly onto the sample addition zone in the case of a fingerstick blood draw. The sample moves by capillary action in the fluid flow path through an optional filter, and into the reagent zone where it dissolves one or more reagents. The sample flows away from the reagent zone and into the detection zone.

Next the sample and a dissolved reagent plume moves by capillary action into the detection zone. The detection zone has a length capable of having at least six zones spaced at least a sufficient distance apart in order to discriminate each signal peak from its adjacent signal peak. There a signal representative of the presence or concentration of the analyte(s) or control(s) is produced. In a preferred embodiment the sample or the one or more reagents having a detection element is captured having in the detection zone, such as by antibodies on the surface of the detection zone and a signal representative of the presence or concentration of the analyte(s) or control(s) is produced, such as by reading a signal that is produced by the detection element to determine the presence or concentration of the analyte(s) or control(s). The reader as described above is then used to read the signal that is detected in the detection zone to determine the presence or concentration of the analyte(s). The sample and any other unbound material moves from the detection zone and into the wicking zone. Also, one or more washes may follow the sample through the device to wash any unbound detection element away from the detection zone.

The method, assay device, and reader according to an embodiment of the invention have many advantages, mainly related to the improved reaction kinetics of the immunochemical reactions and the increased sensitivity of the assay.

It is to be understood that this invention is not limited to the particular embodiments shown here. The following examples are provided for illustrative purposes and are not intended to limit the scope of the invention since the scope of the present invention is limited only by the appended claims and equivalents thereof.

### Examples

### Example 1

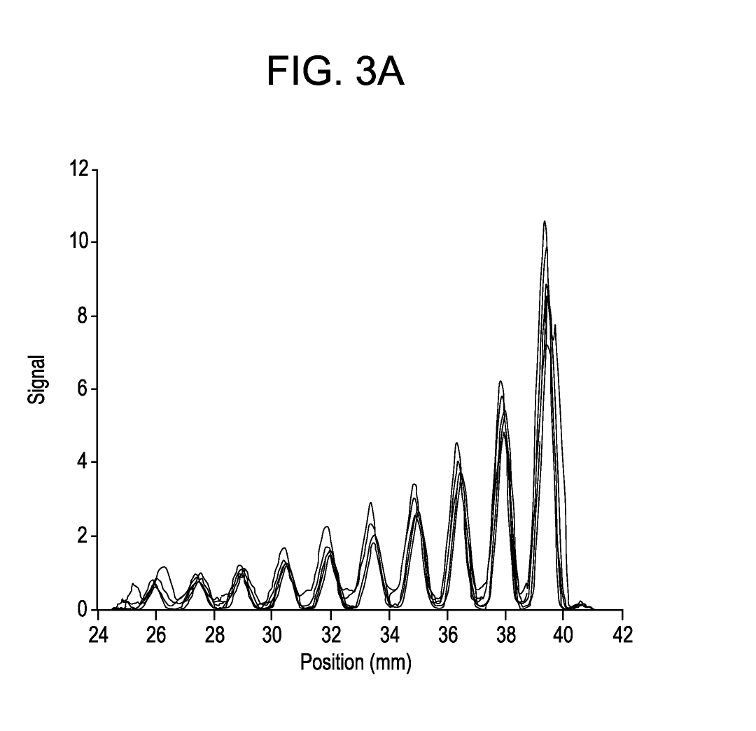

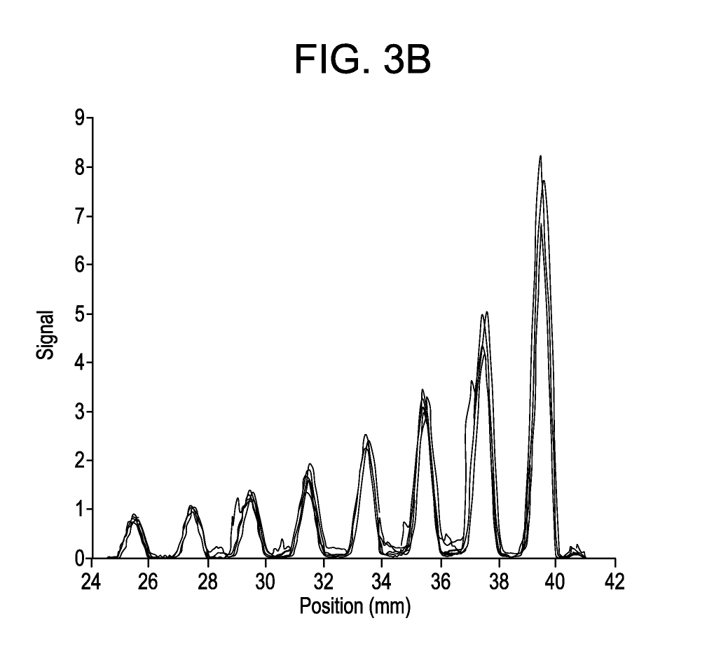

Plastic substrate chips made of Zeonor (Zeon, Japan) having oxidized dextran on the surface for covalently immobilization of proteins via Schiff base coupling were used. Fluorescently labeled Anti-NT-proBNP monoclonal antibody was deposited and dried to create a reagent zone. Anti-NT-proBNP monoclonal antibody was deposited and dried to create a detection zone. A small amount of Triton X-45 was deposited on the device to increase wettability of the sample for better capillary flow. Sample was added to the sample zone of the device and the capillary action of the micropillar array distributed the sample through the flow channel into the wicking zone. A typical assay time was about 10 minutes. The signal intensities from the fluorescently labeled complexes in the detection zone were recorded in a prototype line-illuminating fluorescence scanner. The results from the experiments are shown in FIGS. 2, 3A-B, 4, 7A-B, 8A-C and 9, which have been described above.

### Example 2

Plastic substrate chips made of Zeonor (Zeon, Japan) having oxidized dextran on the surface for covalent immobilization of proteins via Schiff base coupling were used. Fluorescently labeled Anti-NT-proBNP monoclonal antibody was deposited and dried to create a reagent zone. Anti-NT-proBNP monoclonal antibody was deposited and dried to create a detection zone. A small amount of Triton X-45 was deposited on the device to increase wettability of the sample for better capillary flow. Sample was added to the sample zone of the device and the capillary action of the micropillar array distributed the sample through the flow channel into the wicking zone. A typical assay time was about 10 minutes. The signal intensities from the fluorescently labeled complexes in the detection zone were recorded in a prototype line-illuminating fluorescence scanner. The experimental data shown in FIG. 2 was collected using a human serum sample containing 50 pg/mL NT-proBNP.

### Example 3

The experimental data shown in FIG. 3 utilized plastic substrate chips that were prepared similarly to those described above for FIG. 2. Fluorescently labeled Anti-PIGF (Placental growth factor) monoclonal antibody was deposited and dried to create a reagent zone. A second monoclonal antibody was deposited and dried to create 10 discrete detection zones spaced 1.5 mm apart center-to-center in FIG. 3A. 8 Discrete detection zones spaced 2.0 mm apart center-to-center in FIG. 3B. The experimental data shown in FIG. 3 was collected using a human serum sample containing 526 pg/mL PIGF.

### Example 4

The experimental data shown in FIG. 7 utilized reduced volume chips (R2.01a) described in this invention. The NTproBNP reagents utilized in the reagent and detection zones are the same as those described above. The carbamazepine detection reagent was prepared by covalently linking a Carbamazepine hapten and a fluorescent label to bovine serum albumin. The phenobarbital detection reagent was prepared by covalently linking a phenobarbital hapten and a fluorescent label to bovine serum albumin. Monoclonal carbamazepine and phenobarbital antibodies were deposited and dried to create the detection zones. Varying levels of carbamazepine (0, 4, and 16 ug/mL) were spiked into human serum containing 0 ug/mL phenobarbital and 5000 pg/mL NTproBNP to generated the data shown in FIG. 7.

### Example 5

The experimental results shown in FIG. 8 also utilized reduced volume chips (R2.01a) described in this invention. The NTproBNP and PIGF reagents utilized in the reagent and detection zones are the same as those described above. The iPTH (intact parathyroid hormone) reagents were prepared and deposited in a similar manner. Fluids were prepared containing low, mid and high levels of NTproBNP, PIGF, and iPTH by spiking the analytes into human serum. The levels of NTproBNP tested were 0, 1500, and 5000 pg/mL. The levels of PIGF tested were 3, 300, and 2240 pg/mL. The levels of iPTH tested were 0, 300, and 2230 pg/mL

### Example 6

The experimental results shown in FIG. 9 also utilized reduced volume, reduced footprint chips (R3.04). The PHBR and CRBM reagents were the same as described in FIG. 7. The Risperidone reagents were similar to those described for PHBR and CRBM. Five fluids were prepared containing none, low, mid, mid-high, and high of all 3 analytes spiked into human serum. The CRBM levels ranged from 0-18 ug/mL. The PHBR levels ranged from 0-35 ug/mL. The RISP levels ranged from 0-410 ug/mL.

### Example 7

In another set of experiments, a panel of four anti-psychotic drugs (Aripiprazole (ARIP), Olanzapine (OLAN), Quetiapine (QUET) and Risperidone (RISP)), were multiplexed at alternate locations on an assay device having eight reaction zones. In each experiment, conjugates for each of the drugs were deposited in the reagent zone and capture antibodies were deposited on every other zone in the detection zones.

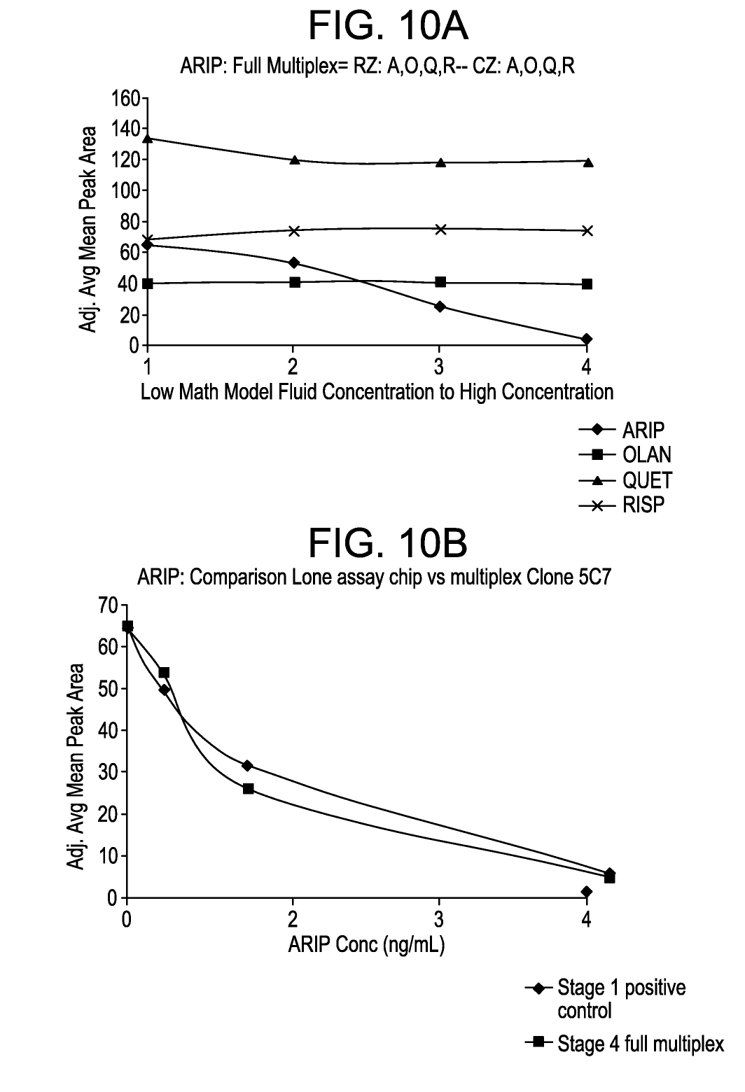

For the first experiment, varying concentrations of ARIP were spiked in human serum and assayed on the device described above. FIG. 10A shows the response curve for all of the drugs. Since all of these are all competitive assays the presence of signal for those drugs not spiked in the sample (i.e., OLAN, QUET, RISP) is expected. The signal for ARIP decreased as the concentration increased as shown in FIG. 10A, again because ARIP is a competitive assay, a higher concentration will result in reduced signal. In FIG. 10B, a dose response curve for ARIP alone in an assay device (shown as diamonds ♦) and multiplexed as described above (shown as squares ▪) is shown. Remarkably, the curves are almost identical. This indicates that the presence of OLAN, QUET and RISP does not affect the sign for ARIP. Thus, FIGS. 10A and 10B show that multiplexing is possible according to the present invention.

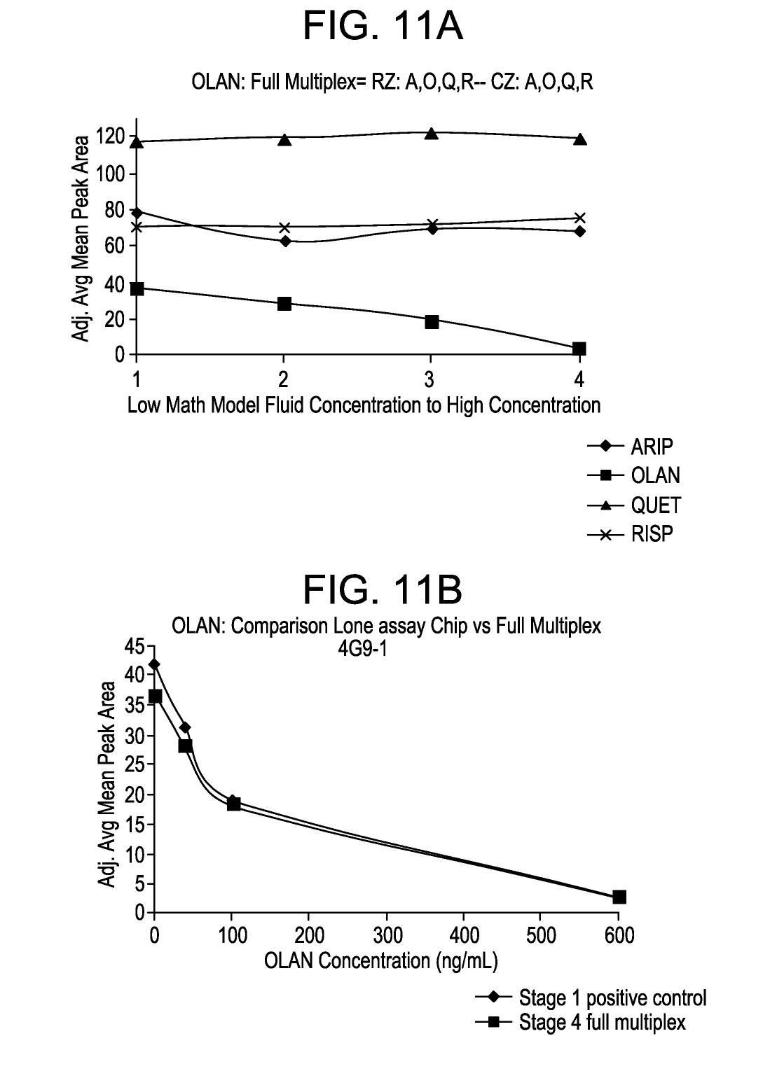

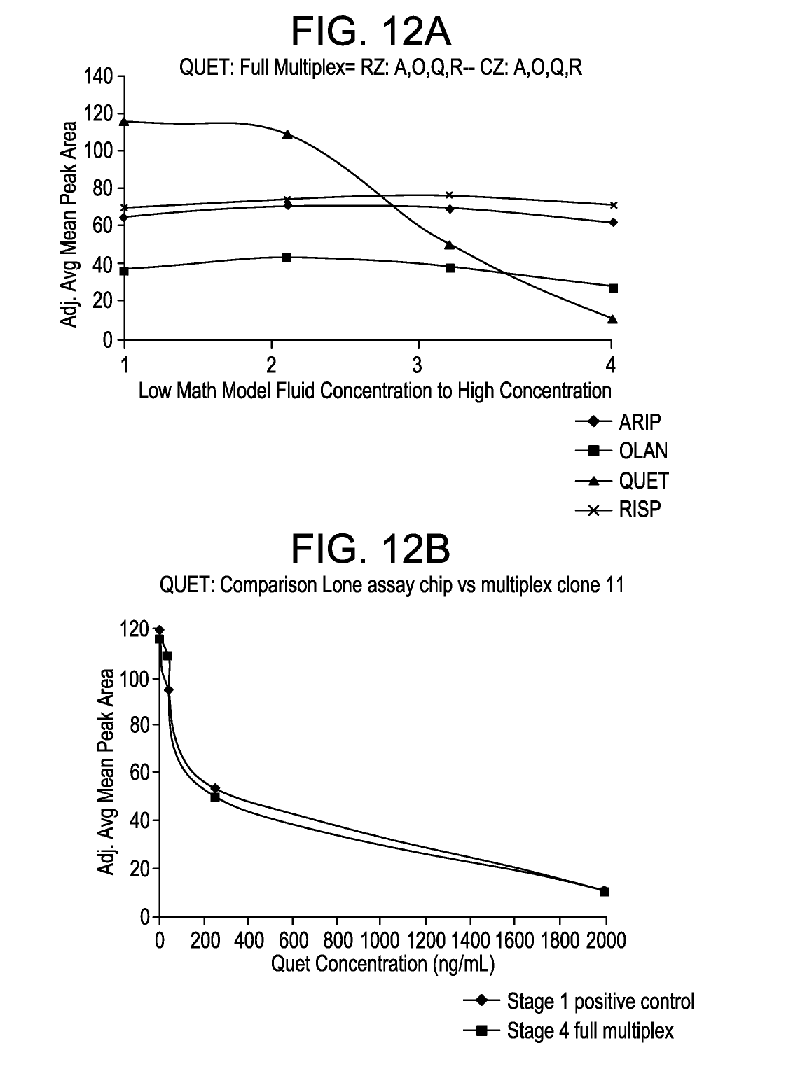

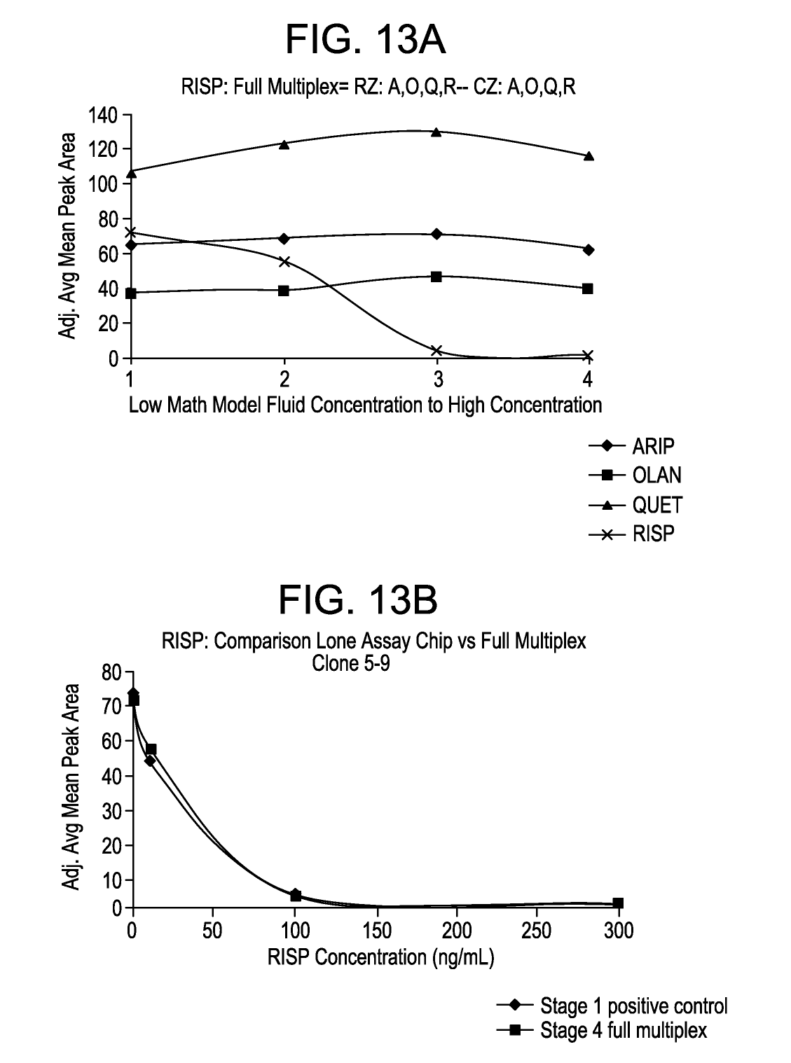

FIGS. 11A and 11B, FIGS. 12A and 12B and FIGS. 13A and 13B show the same results for OLAN, QUET and RISP, respectively.

### Additional Embodiments

1. An assay device for determining the concentration of multiple analytes or controls where the device is capable of determining the presence or concentration of at least six analytes or controls comprising: a fluid flow path; a liquid sample addition zone; a reagent zone downstream and in fluid communication with the sample addition zone containing one or more reagents; multiple detection zones in fluid communication with the reagent zone, wherein the fluid flow path which extends through the detection zones has a length capable of having at least six detection zones linearly spaced at least a sufficient distance apart in order to discriminate each signal peak from its adjacent signal peak; and a wicking zone in fluid communication with the detection zones having a capacity to receive liquid sample flowing from the detection zone, wherein the fluid flow path extends from the sample zone to the wicking zone, and at least a part of the fluid flow path has a substrate and projections which extend substantially vertically from the substrate, wherein the projections have a height, cross-section and a distance between one another that defines a space between the projections capable of generating capillary flow parallel to the substrate surface.

2. An assay device as disclosed in embodiment 1, wherein the multiple detection zones are spaced around at least 1.8 mm apart.

3. An assay device as disclosed in embodiment 2, wherein the multiple detection zones are spaced around at least 2 mm apart.

4. An assay device as disclosed in embodiment 1, 2 or 3, wherein the fluid flow path which extends through the detection zones has a length capable of having at least at least eight zones.

5. An assay device as disclosed in embodiment 4, wherein the fluid flow path which extends through the detection zones has a length capable of having are ten zones.

6. An assay device as disclosed in embodiment 1, wherein the length of flow path through the detection zones is at least 20 mm.

7. An assay device as disclosed in embodiment 1, wherein multiple assay formats are present on the same device.

8. An assay device as disclosed in embodiment 7, wherein at least one of the assays is competitive assay and at least one of the assays is a sandwich-type assay.

9. An assay device as disclosed in embodiment 1, wherein the width of the flow path through the detection zones is in the range of about 0.5 to 1.2 mm.

10. An assay device as disclosed in embodiment 1, wherein total area of the assay device is ≦900 mm².

11. An assay device as disclosed in embodiment 10, wherein total area of the assay device is ≦700 mm².

12. An assay device as disclosed in embodiment 1, wherein the assay device is rectangular and the dimensions of each side are ≦30 mm.

13. An assay device as disclosed in embodiment 12, wherein the assay device is rectangular and the dimensions are approximately ≦24×28 mm.

14. An assay device as disclosed in embodiment 1, wherein the assay device is capable of using a sample size of ≦50 μl.

15. An assay device as disclosed in embodiment 14, wherein the assay device is capable of using a sample size of ≦40 μl.

16. An assay device as disclosed in embodiment 15 wherein the assay device is capable of using a sample size of ≦35 μl.

17. An assay device as disclosed in embodiment 16, wherein the assay device is capable of using a sample size of 25 μl.

18. An assay device as disclosed in embodiment 1, wherein the one or more reagents in the reagent zone comprises labeled conjugate material.

19. An assay device as disclosed in embodiment 1, further comprising a filter.

20. An assay device as disclosed in embodiment 1, further comprising a reagent addition zone.

21. An assay device as disclosed in embodiment 20, wherein the reagent addition zone is before the reagent zone.

22. A method of performing an assay on a liquid sample for the presence or concentration of multiple analytes or controls, on an assay device capable of determining the presence or concentration of at least six analytes or controls, comprising: depositing a liquid sample containing the analyte(s) of interest onto a sample addition zone of an assay device; moving the sample by capillary action through a fluid flow path into a reagent zone where it dissolves one or more reagents; flowing the sample away from the reagent zone having a dissolved reagent plume containing one or more reagents and into multiple detection zones by capillary action through the fluid flow path, wherein the fluid flow path which extends through detection zones has a length capable of having at least six zones spaced at least a sufficient distance apart in order to discriminate each signal peak from its adjacent signal peak, wherein signals representative of the presence or concentration of analyte(s) or control(s) is produced; reading the signals that are produced in the multiple detection zones to determine the presence or concentration of the multiple analytes or controls.

23. A method as disclosed in embodiment 22, wherein the analyte(s) or the one or more reagents having a detection element is captured by capture elements in the detection zone, and a signal representative of the presence or concentration of the analyte(s) or control(s) is detected.

24. A method as disclosed in embodiment 23, wherein the capture element comprises antibodies on the surface of the detection zone.

25. A method as disclosed in embodiment 22, wherein the multiple zones are spaced around at least 1.8 mm apart.

26. A method as disclosed in embodiment 25, wherein the multiple zones are spaced around at least 2 mm apart.

27. A method as disclosed in embodiment 22, 25, 26, wherein the fluid flow path which extends through the detection zones has a length capable of having at least eight zones.

28. A method as disclosed in embodiment 27, wherein the fluid flow path which extends through the detection zones has a length capable of having at least ten zones.

29. A method as disclosed in embodiment 22, wherein the length of the flow path through the detection zones is at least 20 mm.

30. A method as disclosed in embodiment 22, wherein multiple assay formats are present on the same device.

31. A method as disclosed in embodiment 30, wherein the sample moves from the detection zone and into the wicking zone, and the signal may be read immediately or a short time after the sample has moved through the detection zone.

32 A method as disclosed in embodiment 30, wherein one or more washes may follow the sample through the assay device to wash any unbound detection element away from the detection zone.

33. A method as disclosed in embodiment 22, wherein total area of the assay device is ≦900 mm².

34. A method as disclosed in embodiment 33, wherein total area of the assay device is ≦700 mm².

35. A method as disclosed in embodiment 33, wherein the assay device is rectangular and the dimensions of each side are ≦30 mm.

36. A method as disclosed in embodiment 35, wherein the assay device is rectangular and the dimensions are approximately ≦24×28 mm.

37. A method as disclosed in embodiment 22, wherein the assay device is capable of using a sample size of ≦50 μl.

38. A method as disclosed in embodiment 37, wherein the assay device is capable of using a sample size of ≦40 μl.

39. A method as disclosed in embodiment 38, wherein the assay device is capable of using a sample size of ≦35 μl.

40. A method as disclosed in embodiment 39, wherein the assay device is capable of using a sample size of 25 μl.

Those skilled in the art will appreciate that the invention and embodiments thereof described herein are susceptible to variations and modifications other than those specifically described. It is to be understood that the invention includes all such variations and modifications. The invention also includes all of the steps and features referred to in this specification, individually or collectively, and any and all combinations of any two or more of the steps or features.

Co-pending applications entitled “Low Volume Assay Device Having Increased Sensitivity” (Application No. 61/588,758, Attorney Docket No. CDS 5111 USPSP, first named inventor: Phil Hosimer), “Assay Device Having Multiple Reagent Cells” (Ser. No. 61/588,738, Attorney Docket No. CDS5104USPSP, first named inventor Zhong Ding), “Assay Device Having Uniform Flow Around Corners” (Application No. 61/588,745, Attorney Docket No. CDS5110USPSP, first named inventor James Kanaley), “Controlling Fluid Flow Through An Assay Device” (Application No. 61/588,772, Attorney Docket No. CDS5112USPSP, first named inventor James Kanaley), and “Assay Device Having Controllable Sample Size” (Application No. 61/588,899, Attorney Docket No. CDS5114USPSP, first named inventor, Ed Scalice), all filed Jan. 20, 2012 and all incorporated by reference in their entireties.

What is claimed is:

1. An assay device for determining the concentration of multiple analytes or controls where the device is capable of determining the presence or concentration of at least six analytes or controls comprising:
a fluid flow path;
a liquid sample addition zone;
a reagent zone downstream and in fluid communication with the sample addition zone containing one or more reagents;
multiple detection zones in fluid communication with the reagent zone, wherein the fluid flow path which extends through the detection zones has a length capable of having at least six detection zones linearly spaced at least a sufficient distance apart in order to discriminate each signal peak from its adjacent signal peak; and
a wicking zone in fluid communication with the detection zones having a capacity to receive liquid sample flowing from the detection zone,
wherein the fluid flow path extends from the sample zone to the wicking zone, and at least a part of the fluid flow path has a substrate and projections which extend substantially vertically from the substrate, wherein the projections have a height, cross-section and a distance between one another that defines a space between the projections capable of generating capillary flow parallel to the substrate surface.

2. An assay device as claimed in claim 1, wherein the multiple detection zones are spaced around at least 1.8 mm apart.

3. An assay device as claimed in claim 2, wherein the multiple detection zones are spaced around at least 2 mm apart.

4. An assay device as claimed in claim 1, wherein the fluid flow path which extends through the detection zones has a length capable of having at least at least eight zones.

5. An assay device as claimed in claim 4, wherein the fluid flow path which extends through the detection zones has a length capable of having are ten zones.

6. An assay device as claimed in claim 1, wherein the length of flow path through the detection zones is at least 20 mm.

7. An assay device as claimed in claim 1, wherein multiple assay formats are present on the same device.

8. An assay device as claimed in claim 7, wherein at least one of the assays is competitive assay and at least one of the assays is a sandwich-type assay.

9. An assay device as claimed in claim 1, wherein the width of the flow path through the detection zones is in the range of about 0.5 to 1.2 mm.

10. An assay device as claimed in claim 1, wherein total area of the assay device is ≦900 mm².

11. An assay device as claimed in claim 1, wherein the assay device is rectangular and the dimensions of each side are ≦30 mm.

12. An assay device as claimed in claim 1, wherein the assay device is capable of using a sample size of ≦25 μl.

13. An assay device as claimed in claim 1, wherein the one or more reagents in the reagent zone comprises labeled conjugate material.

14. A method of performing an assay on a liquid sample for the presence or concentration of multiple analytes or controls, on an assay device capable of determining the presence or concentration of at least six analytes or controls, comprising:
depositing a liquid sample containing the analyte(s) of interest onto a sample addition zone of an assay device;
moving the sample by capillary action through a fluid flow path into a reagent zone where it dissolves one or more reagents;
flowing the sample away from the reagent zone having a dissolved reagent plume containing one or more reagents and into multiple detection zones by capillary action through the fluid flow path, wherein the fluid flow path which extends through detection zones has a length capable of having at least six zones spaced at least a sufficient distance apart in order to discriminate each signal peak from its adjacent signal peak, wherein signals representative of the presence or concentration of analyte(s) or control(s) is produced;
reading the signals that are produced in the multiple detection zones to determine the presence or concentration of the multiple analytes or controls.

15. A method as claimed in claim 14, wherein the analyte(s) or the one or more reagents having a detection element is captured by capture elements in the detection zone, and a signal representative of the presence or concentration of the analyte(s) or control(s) is detected.

16. A method as claimed in claim 15, wherein the capture element comprises antibodies on the surface of the detection zone.

17. A method as claimed in claim 14, wherein the multiple zones are spaced around at least 1.8 mm apart.

18. A method as claimed in claim 17, wherein the multiple zones are spaced around at least 2 mm apart.

19. A method as claimed in claim 14, wherein the fluid flow path which extends through the detection zones has a length capable of having at least eight zones.

20. A method as claimed in claim 19, wherein the fluid flow path which extends through the detection zones has a length capable of having at least ten zones.

21. A method as claimed in claim 14, wherein the length of the flow path through the detection zones is at least 20 mm.

22. A method as claimed in claim 14, wherein multiple assay formats are present on the same device.

23. A method as claimed in claim 14, wherein the assay device is capable of using a sample size of ≦25 μl.
